(19) United States
(12) Patent Application Publication
(10) Pub. No.: US 2005/0171501 A1
(43) Pub. Date: Aug. 4, 2005

(54) Intravenous solution producing systems and methods

(76) Inventor: Thomas Kelly, Tampa, FL (US)

Correspondence Address: Joseph P. Reagen, Esq.;Corporate Counsel, Renal Division, Baxter Healthcare Corporation, One Baxter Parkway, DF3-3E, Deerfield, IL, 60015-4633, US

(21) Appl. No.: 11/025,008

(22) Filed: Dec. 28, 2004

Related U.S. Application Data
(60) Provisional application No. 60/541,858, filed on Feb. 3, 2004.

(51) Int. Cl. A61M 31/00

(52) U.S. Cl. 604/500

(57) ABSTRACT

A portable system is provided in one embodiment that combines stages of water purification to produce sterile pyrogen free water for injection. It also adds NaCl to the purified water to produce sterile and pyrogen free saline for injection in a relatively compact and mobile delivery package. The solution produced is then bagged via suitable bagging equipment. Various methods are also provided to deareate the solution, ensure its stability and to disinfect the solution between uses and prior to use. The systems and methods in one embodiment produce on-line saline of less than 0 cfu and less than 0.03 EU/ml, while meeting acceptable levels of other chemical contaminants, heavy metals and organics.

### PRIORITY CLAIM

This application claims priority to and the benefit of U.S. Provisional Patent Application No. 60/541,858, filed Feb. 3, 2004, entitled “Intravenous Solution Producing Systems And Methods”, the entire contents of which are hereby incorporated by reference and relied upon.

### BACKGROUND

The present invention relates generally to intravenous solutions and more particularly for an on-line method and apparatus of producing same.

Known intravenous solution bags are made in a facility and shipped to the point of use. The safeguards in making and bagging the solution are stringent and closely controlled. The packaging of the bagged solution is also performed carefully so that the integrity of the solution is not compromised. Additionally, the packaged solution bags are shipped under conditions so that the solution remains sterile or of an injectable quality. In short, the bagging, packaging and shipping of sterile solution bags ads significantly to the cost of the bag to the end user.

In particular, the remote bagging of sterile solution is time consuming and expensive. Intravenous solution bags have been filled in clean rooms under hoods that purify the air within specified limits. The bags are sterilized before being taken into the room to prevent the room from being contaminated. The bags are then re-sterilized after being filled to ensure sterility of the filled bag. Certain types of solutions are damaged by the sterilization process. Those bags must be processed in an even heightened sterile environment to prevent contamination during the filling and sealing processes to eliminate the second sterilization step. Clean rooms capable of maintaining the necessary sterile environment are expensive to build and operate. Further, the rooms require everything in the room, including workers and equipment, to be sterilized an/or disinfected.

For hospitals and other healthcare facilities, there is a need from a cost standpoint to have a system and method for producing sterile solution closer to the point to use, e.g., closer to a hospital. Indeed, to save cost it would be desirable for hospitals and other healthcare facilities to make and bag their own supply of intravenous solution, rather than to have such solution delivered.

Besides the cost of packaging and delivering the solution, the shear weight of the solution bags can become a problem in their shipment and delivery. For example, in disaster situations or in the battlefield where significant volumes of sterile pyrogen-free solution is required, it can become difficult to deliver the necessary amount of fluid in time. The weight of the fluid requires the use of heavy machinery or aircraft to deliver any significant amount of sterile fluid. Moreover, in an emergency or battlefield situation certain conventional routes of transportation, such as by truck or railroad may not be possible, limiting the mode of transport to air transport, which is expensive and not the most efficient mode. Indeed, certain situations may make it impossible to deliver large quantities of commercially produced saline (or sterile water for injection) due to location, inaccessibility or logistics.

In the above situations it would be desirable to have a mobile, rugged device capable of producing a sterile and pyrogen-free solution and of bagging the solution on-line. Indeed, there are multiple uses for a system and method that produces in a relatively short period of time, from an available water source, such as a water tap, an on-line and ready supply of pyrogen-free bagged solution, i.e., water suitable for injection, saline or lactated ringers.

### SUMMARY

The present invention enables the production of sterile or injectable quality water for injection and sterile pyrogen free saline or located ringers at the point of use in a small compact package. The solution produced has low levels of chemical contaminants, heavy metals and organics.

To that end, a system and method are provided for producing an on-line and ready supply of pyrogen bagged solution, i.e., water suitable for injection, saline, dextrose-saline or lactated ringers. The apparatus and method are useful at hospitals. The system and method are also useful in disaster situations, where significant volumes of sterile pyrogen-free solution are required, but where it is not possible to send in large quantities of commercially produced saline (or sterile water for injection) due to location, inaccessibility, or logistics. A mobile, rugged device capable of producing bagged sterile and pyrogen-free solution is provided for such situations.

The invention provides in one embodiment a portable device that combines stages of water purification to produce sterile pyrogen free water for injection. The device also adds NaCl to the purified water to produce sterile and pyrogen free saline for injection in a relatively compact and mobile delivery package. Various methods are also provided to deareate the solution, ensure its stability and to disinfect the solution between uses and prior to use. The solution produced is then bagged via suitable bagging equipment. The systems and methods in one embodiment produce saline of 0 cfu and less than 0.03 EU/ml, while meeting acceptable levels of other chemical contaminants, heavy metals and organics.

The system in one embodiment is intended for use in crisis, disaster and battlefield scenarios. Due to the nature of those types of environments, the system is robust enough to withstand shock waves and considerable electrical noise, while functioning properly. The system operates over a wide range of voltages and can withstand severe voltage fluctuations as well as overcurrent and electrostatic discharge (“ESD”) events.

It is therefore an advantage of the present invention to create medical grade solutions where it may be impossible or cost prohibitive to ship commercially produced solutions.

It is another advantage of the present invention to provide a system that selectively produces different types of bagged solutions, such as saline, lactated ringer or dextrose NaCl.

It is a further advantage of the present invention to provide an injectable solution producing system that includes an on-board disinfecting feature.

It is yet another advantage of the present invention to provide an injectable solution producing system that is controllable locally or remotely.

It is still a further advantage of the present invention to provide an injectable solution producing system that is small enough to be readily transported by air, ship, train or vehicle.

It is still another advantage of the present invention to provide an injectable solution producing system that is rugged enough to be moved in an emergency or battlefield application, such as being dropped to a use point from an airplane.

Further still, it is an advantage of the present invention to provide an injectable solution producing system that is scalable to produce varying daily outputs of bagged solution as desired.

Additional features and advantages of the present invention are described in, and will be apparent from, the following Detailed Description of the Invention and the figures.

### BRIEF DESCRIPTION OF THE FIGURES

FIG. 1 is a schematic diagram illustrating one embodiment of a system of the present invention for producing injectable quality solutions.

FIG. 2 is a schematic diagram illustrating one embodiment of the bag of the present invention.

FIG. 3 is a schematic diagram illustrating one embodiment of a rinse/disinfect feature for the injectable quality solution producing system of the present invention.

FIG. 4 is a schematic diagram illustrating one alternative embodiment for the injectable quality solution producing system of the present invention.

FIG. 5 is a schematic diagram illustrating another embodiment of the bag and associated apparatus of the present invention.

FIG. 6 is a schematic diagram illustrating a further embodiment of the bag and associated apparatus of the present invention.

FIG. 7 is a schematic diagram illustrating a further alternative embodiment for the injectable quality solution producing system of the present invention.

FIGS. 8 to 10 are process flow diagrams further illustrating the systems, methods and apparatuses of the present invention.

FIG. 11 is a more detailed view of particular components described in connection with and shown in FIG. 1.

### DETAILED DESCRIPTION

There is a need to be able to produce an injectable quality or sterile and pyrogen free bagged solution (“solution” as used herein refers generally to water suitable for injection, saline, lactated ringers, dextrose/NaCl unless otherwise specified) on-line from a relatively small, portable unit. It is desirable to have a system that can be used in hospitals under “normal” use situations as well as in other places undergoing crisis or disaster situations, where significant volumes of sterile pyrogen-free solution are needed, but where it is not possible to deliver large quantities of commercially produced solution due to location, inaccessibility, or logistics. The present systems and methods answer those needs.

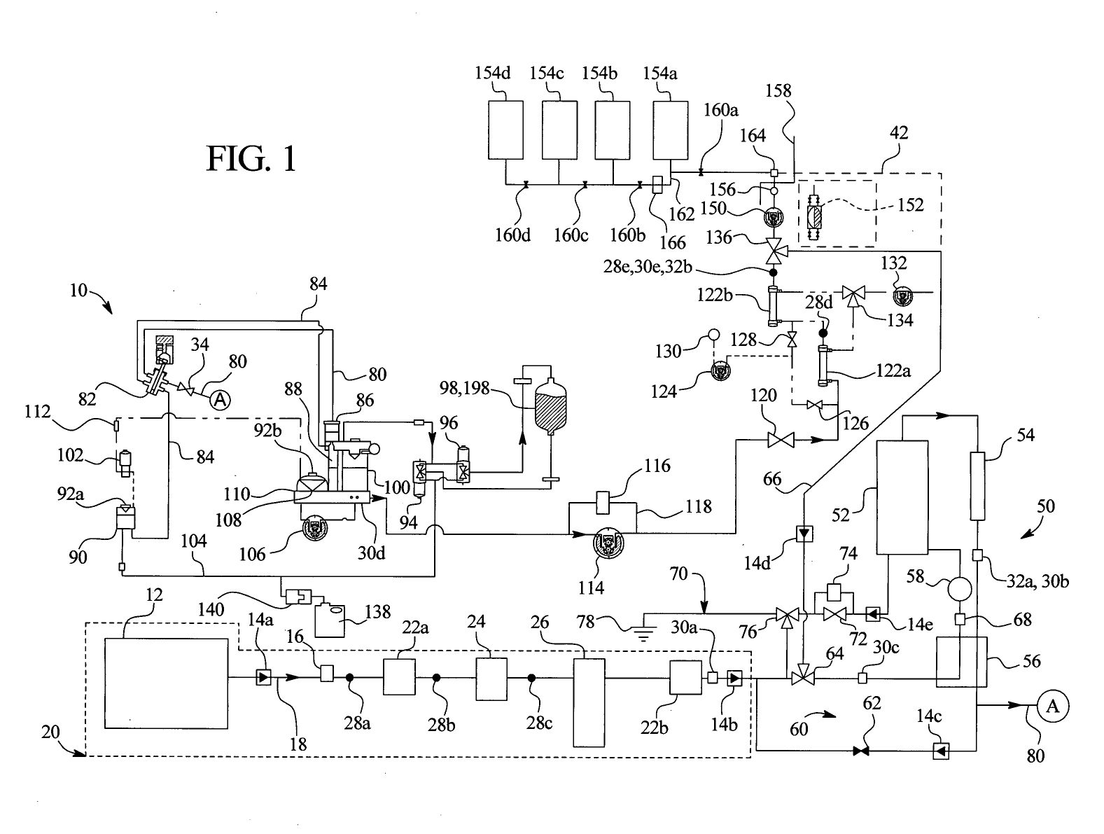

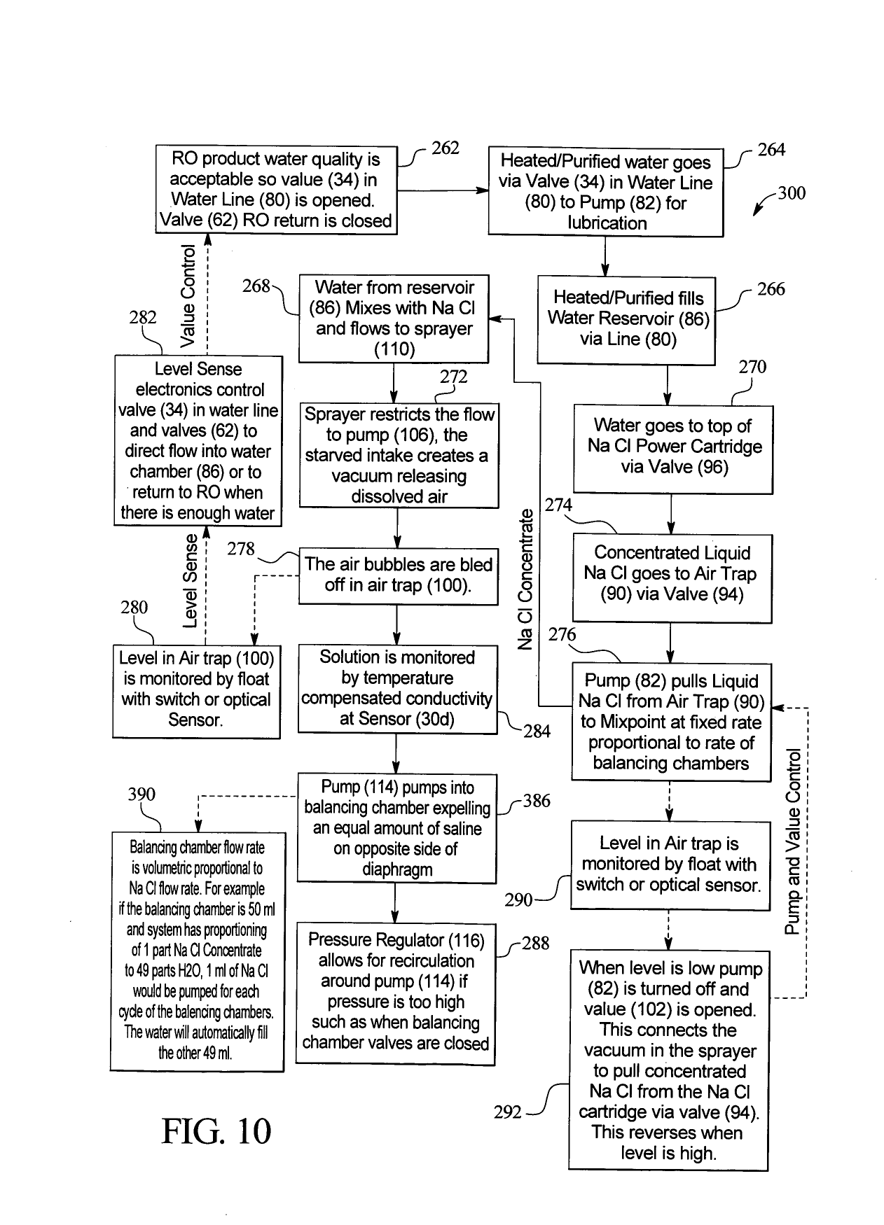

Referring now to the drawings and in particular to FIGS. 1 and 8, a system 10 and method 200 are illustrated simultaneously. System 10 is provided in a portable and sturdy enclosure in one embodiment, which is adapted to be moved to a disaster site, a war zone or other remote area. System 10 can otherwise be skid mounted or configured to be located in a hospital, doctor's office or other medical facility. System 10 is capable of producing sterile and pyrogen free solutions on-line that can be bagged at the point of use. In one embodiment, system 10 makes enough solution for at least twenty-four hours of use, e.g., the output of system 10 per day is enough to supply the solution needed per day. System 10 is also scalable depending on the amount of bagged sterile solution required.

### Pretreatment Water Purification Unit

System 10 includes at steps 202 and 204 a pretreatment unit 20. Pretreatment unit 20 includes a water source 12, which can be a container of water, an on-line source, a tap or a natural source, such as a lake or river. In one preferred embodiment, potable water is used. If it is not possible to connect to a suitable direct source or tap, source 12 includes a container into which supply water can be added. In a battlefield or emergency situation it is possible that suitable supply water is trucked-in, air freighted in or dropped in from an aircraft.

A backflow preventer, e.g., check valve 14a, is provided to prevent flow from flowing back to source 12. A water on/off valve 16 is also provided in the water input line 18, which allows and stops the flow of inlet water from source 12 through the line 18. On/off valve 16 is manual, electrically or pneumatically controlled as desired.

Water flows through an optional filter 22a, such as a five to ten micron particle filter. Filter 22a removes sediments and gross particulates from the incoming water. Water flows from particulate filter 22a to a carbon filter 24. Carbon filter 24 removes chemicals, such as chlorine, chloramines and organic substances from the water. Next, the water flows through an optional deionization unit 26, such as a mixed bed deionization unit that uses cationic and anionic deionization resins to form neutral water. Deionization unit 26 removes chemical contaminants as well as ions from the water. Deionization unit 26 may be optional assuming the reverse osmosis (“RO”) unit described below is provided and system 10 is configured to operate only if the RO unit is working properly. Therefore, the RO unit can be checked by checking the RO membrane based on circulation of a known purified/NaCl solution.

A second five to ten micron particle filter 22b is located downstream from deionization unit 28. Filter 22b is important because filter 22b removes carbon particles or resin beads that have come free from carbon filter 24 or deionization unit 26, respectively.

Pressure sensors 28 (collectively referring to sensors 28a, 28b, 28c, etc.) are provided at various locations along inlet line 18 to determine the life of the particle and carbon filters. Pressure sensors 28 are optional if time of use or volume of water flowed is used instead as the method to determine the life of filters 22a and 22b, 24 and 26.

A first pressure sensor 28a is placed between valve 16 and a filter 24. Pressure sensor 28a detects the presence or absence of water flowing from source 12 through line 18 and/or if filter 22a is too full of trapped particulate. Sensors 28b and 28c likewise sense for flow blockages in carbon filter 24 and deionization unit 26, respectively.

A conductivity measuring device 30a, which can be temperature compenstated, is provided to measure the performance of the mixed bed deionization unit 26. Conductivity measuring device 30a sends a signal to a conductivity monitor (not illustrated), for example located at a control panel, which displays information on the quality of the output water from the deionization unit 26. The control system can be set to provide an alarm if the conductivity of the liquid is out of range, e.g., if the water post deionization unit 26 has a resistance of less than one mega-Ohm. Such an alarm signals that the deionization unit cartridge needs to be replaced or that that one of its resin beds is exhausted. In an alternative embodiment, a self-regenerating deionization unit 26 is used instead of a replaceable mixed bed deionization unit 26.

A backflow preventer or check valve 14b is placed downstream from conductivity measuring device 30a in the illustrated embodiment. Each of the above-described components for pretreatment unit 20 can be rack and shock mounted. Any of those components requiring replacement is thereby easily replaced.

### Water Treatment Unit

Water treatment unit 50 at step 206 includes a reverse osmosis (“RO”) membrane unit 52, heater 54 (optional), heat exchanger 56, at least one pump 58 and associated plumbing. Water flows out of deionization unit 26, flows through heat exchanger 56, which pre-heats the water prior to reaching RO unit 52 to improve the efficiency of unit 52. Feed pump 58 supplies water under pressure to RO unit 52. Water exiting RO unit 52 flows to heater 54, which heats the water to, e.g., approximately 85° C., as indicated by temperature sensor 68. Heater 54: (i) pasteurizes the water; (ii) preheats via heat exchanger 56 the water entering RO unit 52; and (iii) creates bubbles in the water, which are bled off in an air trap 90 (discussed below) to dearate the water.

Heater 54 and heat exchanger 56 are optional in alternative embodiments employing cold disinfection. Heater 54 is alternatively located prior to the temperature sensor and the feed pump on the inlet to the reverse osmosis device. In that alternative, the pasteurization cycle is absent, however, an ultraviolet light source may be emitted through the product water downstream of the reverse osmosis to reduce contamination, for example, by killing bacteria residing in the product water. In the illustrated embodiment, a temperature sensor 32b and conductivity sensor 30b are located downstream from heater 54.

Water treatment unit 50 includes at step 218 a recirculation feature that increases the efficiency of the system. When downstream demand for water does not require an output from RO unit 52 or the RO water quality is outside of the acceptable range, the excess product can be circulated from after the output of RO unit 52 back to the input of RO unit 52 via recirculation loop 60 to reduce the load on the membranes. Recirculation loop 60 includes a check valve 14c and an overflow valve 62. Valve 62 is opened and closed to activate and deactivate, respectively, the recirculation loop. Valve 34 in line 80 is closed when valve 62 is opened. Valve 34 is opened when valve 62 is closed. Alternatively, valves 34 and 62 could be replaced with a three-way valve.

RO unit 52 uses filtration that is pressurized to overcome the osmotic pressure of the solutes being removed from the water. The RO filtration typically removes chemical contaminants at a rate of about 95% to about 99% and microbiological components, including bacteria, endotoxin and viruses, at a rate of about 99%.

System 10 employs percent rejection monitoring (% rejection=1−(product/input))×100) to verify the effectiveness of the RO membranes prior to filling of the bags with saline. Deionization unit 26 produces water with relatively high resistivity, which makes measuring the RO output difficult using the percent rejection method. A percent rejection valve 64 is therefore added so that saline can be diverted (from a point after the addition of NaCl and before the filling of the bags) via a return line 66 to valve 64, located in the inlet line of RO unit 52. The 0.9% saline produced has a specific conductivity of approximately 15.87 mS/cm plus or minus the proportioning tolerance of the system. The saline is run through the RO unit 52, after which the output conductivity sensor 30b senses the conductivity of the outputted saline, which should be an expected conductivity given that the inputted saline has a known ionic strength. The RO unit's conductivity output and the expected conductivity output are then compared, enabling the corresponding percent rejection to be monitored on a continuous, semi-continuous or periodic basis.

A check valve 14d is placed in return line 66 in one embodiment. A third conductivity sensor 30c, located downstream from valve 64, ensures that the resistivity of the fluid flowing to RO unit 52 is suitable for being tested using the percent rejection method. The resulting percent rejection method of the present invention verifies the RO unit's ability to remove contaminants against the expected results from the saline solution, which has a known ionic strength, and provides a check of the performance of the membrane within RO unit 52.

If the measured percent rejection is acceptable, the product water resistivity measured via sensor 30b is used as an alarm setting until the next check is made. Checks can be updated at any suitable interval, such as when system 10 is idling flow to switch from filling one bag to another. In that way the RO unit 52 performance is monitored systematically and periodically. If the measured percent rejection is not acceptable, the water is rejected through line 70, including check valve 14e, valve 72, regulator 74, disinfect valve 76 to drain 78. Three-way disinfect valve 76 enables rejected product from RO unit 52 to be circulated back to the inlet of unit 52. Reusing rejected product reduces the load on the membranes within unit 52 as does overflow loop 60. In addition, valve 76 allows system 10 to be placed in a recirculation mode, for example, for heat disinfection and citric heat disinfection.

### Saline Preparation

At step 208, product valve 62 is closed and valve 34 is opened, allowing product water exiting RO unit 52 to flow through lubrication line 80 to an additive supply via an NaCl pump 82. The electrolyte mixing, proportioning, confirmation and solution deareation step 208 is shown in more detail in scheme 260 of FIG. 9. In one embodiment, pump 82 is a rotating, reciprocating ceramic piston pump. That type of pump is advantageous because it is set mechanically to fail closed, negating the possibility of a free flow condition. That type of pump requires lubrication, preferably with a clean or sterile lubricant. Product water exiting RO unit 52 without salt is well-suited for such duty as seen at step 262 of FIG. 9. The water lubricates the shaft of ceramic pump 82 as seen at step 264 of FIG. 9. A peristaltic pump, diaphragm pump or gear pump is used alternatively to the ceramic pump 82. In those cases, the flow path of system 10 would change accordingly, e.g., would not need a separate lubrication line 80. Water flowing through pump 82 via lubrication line 80 eventually reaches receptacle 86.

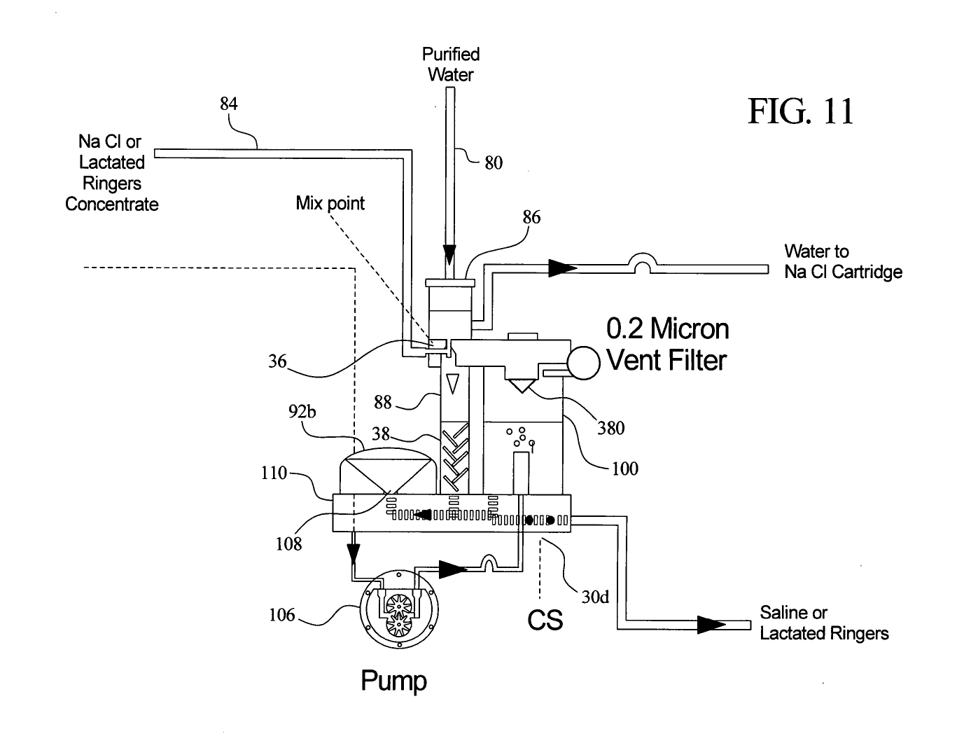

Pressurized water from the RO unit 52 flows via pump 82 through line 80 to water chamber 86 as seen at step 266 of FIG. 9. FIG. 11 illustrates receptacle 86, air trap 100 and other associated components in more detail. Water from chamber 86 flows and mixes with a concentrated sodium chloride (“NaCl”) solution delivered via a line 84 as seen at step 268. In addition, water chamber 86 provides a source of water to an NaCl cartridge 98 to prepare the NaCl solution through either valve 94 or 96. Valves 94 and 96 are each three-way solenoid valves in one embodiment. A combination of two-way valves could be used alternatively in place of valves 94 and 96 as seen at step 270. NaCl cartridge 98 in one embodiment contains dry pharmaceutical grade NaCl powder. System 10 senses when a new cartridge is installed into the saline machine using in one embodiment a switch (not illustrated) on the cartridge holder. That switch may be an optical, reed, micro or any other suitable type of switch that can sense the opening of the cartridge holder.

After cartridge 98 is installed, water reservoir 86 is connected to the inlet of cartridge 98 via valve 94 to enable concentrated liquid NaCl to flow to air trap 90 as seen at step 274. Valve 96 connects the outlet of the cartridge 98 via line 104 to air trap 90. A level control sensor 92a, such as an optical level sensor, may be used at the top of the air trap 90. Sensor 92a is alternatively a reed or Hall effect switch that operates with a float assembly. When sensor 92a senses that the water level in trap 90 is too low, as seen at step 290, an air trap vent valve 102 (in phantom air line) opens momentarily, while sprayer assembly 110 is under a vacuum, which pulls concentrated NaCl solution from cartridge 98, refilling air trap 90 as seen at step 292. As the water fills from the bottom of cartridge 98, air is vented from the cartridge.

If the level in the air trap 90 is too low, pump 82 is turned off temporarily. Air trap 90 is then connected fluidly to the sprayer assembly 110 via the air vent valve 102 and the flow restrictor 112. Sprayer 110 is under a vacuum created by pump 106, which is currently starved for inlet fluid, so that air bubbles are vented off through air trap 100 as seen at step 272. When the level in the air trap 90 is restored to a proper or desired level, air valve 102 closes and pump 82 is restarted.

After a period of time necessary to fill at least a portion of the cartridge 98 with water, valves 94 and 96 change state. Now in a run position, valve 96 enables water to be fed from water chamber 86 to the top of cartridge 98. The output of cartridge 98 flows through valve 94, which is now also in a run state, to air trap 90. Solution flowing out of cartridge 98 is a mixture of NaCl and water and may or may not be a saturated NaCl-solution.

Pump 82 pumps a precise amount of NaCl solution from air from air trap 90 to mix with water at a mixing point 36 in a mixing tube 88 located just below water chamber 86 as seen at step 276. That tube includes or provides a torturous path 38 to thoroughly mix the clean water with the NaCl solution, after which the mixed solution flows to sprayer assembly 110 as seen at step 268. Air removal pump 106 pulls the solution through an orifice 108 in the sprayer assembly 110. Air removal pump 106 also pumps fluid at a rate higher than the flow through sprayer orifice 108, creating a nucleation site to bleed air bubbles off in air trap 100 as seen at step 278.

In one embodiment, the solution level in air trap 100 is controlled by an optical sensor 380 as seen at step 280, which is located at the top of air trap 100. The optical sensor 380 is alternatively replaced by a reed or Hall effect switch operating with a float assembly. The electronics associated with level sensor 380 control valve 34 in water line 80 and valve 62 to direct flow into water chamber 86 or return water to the RO unit 52 if enough water is present in air trap 100 as seen in steps 282 and 262.

The proportioning of the NaCl in water is accomplished in one embodiment by adjusting pump 114 in a feedback loop 118 to achieve a particular conductivity, which is measured at conductivity sensor 30d as seen at step 284. That is, pump 114 is sped up or slowed down to maintain proper conductivity at sensor 30d as seen at step 286. Another approach is to volumetrically mix the solution, e.g., to mix a known volume or weight of cleansed water with a known volume or weight of NaCl. Conductivity monitoring provides a confirmation of the solution mixture. Knowing the temperature and assuming the temperature to be constant, the conductivity should accurately reflect the meq/l (the number of grams of a solute contained in one milliliter of a normal solution) of NaCl in the solution.

The controller of system 10 monitors the conductivity output from sensor 30d and calculates the meq/l NaCl continuously as seen at step 284. A thermistor, thermocouple or other type of temperature sensing device is provided with sensor 30d (not illustrated) and is used to compensate for any temperature changes in the NaCl proportioning calculation using conductivity measurements.

When system 10 is turned off, any powder or solution remaining in the cartridge 98 is drained quickly from the cartridge in one embodiment. To activate the drain, a cartridge drain button can be provided on the system's control device, which the operator presses to initiate the drain. A shunt 42 residing between connector 164 and line 66 is activated so that the output of RO unit 52 is shunted though valve 62 to the input of RO unit 52. Pump 82 is run at a high rate of speed to pull the shunted fluid and residual powder or solution quickly from the cartridge. Valve 134 is opened and in fluid communication with the input of filter 122a and line 66, enabling the flushed concentrated solution to be sent through valve 76 to drain line 70.

Pump 114 and pressure regulator 116, located in a bypass line 118 around pump 114, are provided to pump a pressure regulated solution from air trap 100 to a bubble test valve 120. Pressure regulator 116 allows for recirculation around pump 114 if pressure of the solution is too high as seen in step 288. Pump 114 may be any suitable type of fluid pump, such as gear pump.

The concentration proportioning of NaCl may be performed using conductivity feedback as described above, or via a volumetric approach, which is shown in alternative scheme 300 of FIG. 10. Each of the steps of the scheme 300 is the same as in scheme 260 except that step 286 of scheme 260 is changed to step 386 in scheme 300. Step 390 is also added to scheme 300. The volumetric approach would employ a volumetric device (such as device 152 described below) located downstream of the pump 114. The volumetric device could be a balancing chamber, which is configured with two matched cavities separated by a diaphragm. The balancing chamber would use two valves located at its inlet and two valves located at its outlet to control flow to both sides of the diaphragm to expel an amount from one side of the diaphragm that is equal to the amount inputted in the other side of the diaphragm as seen at step 386. The balancing chamber is described in more detail below in connection with alternative flow meter 152.

If volumetric proportioning of NaCl solution is employed, pump 82 in one embodiment is run at a speed that is proportional to the output of the above-described volumetric device. For instance, for a desired 0.9% NaCl solution, pump 82 is operated to pump to the volumetric device at a rate of 0.9% of the volume of the output the device. Sterile or injectable quality water is then made available to the volumetric device to proportionally fill the rest of the cavity defined by the volumetric device as seen at step 390.

When bubble test valve 120 is opened, NaCl solution flows to two ultrafilters 122a and 122b (collectively filters 122) located in series. Each ultrafilter is capable of a significant log reduction of bacteria and endotoxin, such that if one of the filters fails, the solution bags are still filled with solution that is both of an injectable quality and contains less than United States Pharmacopoeia (“USP”) rated levels of endotoxin. To that end, system 10 should be capable of producing saline of about zero culture forming units (“cfu”) and less than 0.03 endotoxin units per milliliter (“EU/ml”), while meeting acceptable levels with regard to other chemical contaminants, heavy metals and organics, which are removed primarily by the pretreatment unit 20 and RO unit 52. It should be appreciated that system 10 thereby produces bags 154 of injectable quality solution utilizing sterile or non-sterile additives, along with downstream ultrafilters to remove virtually, if not all, bacteria and endotoxin from the injectable quality solution.

Filters 122 are reusable filters in one embodiment, which remove remaining bacteria and endotoxin from the NaCl solution prior to filling the supply bags. If filters 122 are reusable, they should be tested prior to use. One suitable reusable ultrafilter is a Medica™ Diapure™ 28 filter. In another embodiment, ultrafilters 122 are single use replaceable filters. One suitable single use ultrafilter is a Medica™ 150u filter. The term “ultrafilter” as used herein includes filters having a membrane pore or membrane opening diameter or length of about 10 to about 1000 Angstroms (“Å”), which effectively filters particles such as endotoxins (pyrogen), viruses and proteins. In one preferred embodiment, the ultrafilters used in the present invention have a range of pore sizes of about 10 to about 40 Å.

One way to check the integrity of ultrafilters 122 is using a bubble point test. In that test, the membranes inside filters 122 are wetted and one side of those membranes is pressurized with air after which the pressure drop is observed. Normally, the pressure decays slowly because air is forced to diffuse through to the other side of the membranes. If even a single fiber is broken or cracked, for example, where the fibers are fitted into a potting material, the pressure decays much more rapidly. The test can be run prior to use and/or intermittently during the filling process.

During the bubble point test, bubble test valve 122 is closed. An air pump 124 is connected by a pair of valves 126 and 128 to the main flow just prior to filters 122a and 122b respectively. At appropriate times, e.g., before, during or after the integrity pressure tests, air pump 124 pulls air from ambient and through an air filter 130, which is a 0.2 micron vent filter in one embodiment. A purge pump 132 operates through a three way valve 134 to pull a vacuum through ultrafilters 122, enabling the corresponding pressure decay to be monitored via pressure sensor 28d (for filter 122a) at step 210 and sensor 28e (for filter 122b) at step 212 to determine if the filters are intact.

While the drawing illustrates air pump 124 on the pre-side of filters 122, an alternative embodiment infuses air from the post-filter side. In the post-filter arrangement, an additional pump (not illustrated) is used to draw the vacuum needed to verify the appropriate pressure decay. Regardless of the configuration of components used for the integrity test if one or more of the filters 122 fails the pressure decay test, the defective filter is changed prior to further use in saline production and the entirety of system 10 is disinfected.

Purge valve 134 and pump 132 enable a portion of the incoming solution to flow along the length of one of the two filters 122 to prevent the build-up of bacteria and/or endotoxin on the outside of the membranes located inside the filters. Three-way purge valve 134 alternates positions sequentially and continuously to enable one or the other ultrafilter 122a or 122b to be cleansed. Pump 132 is run at a slow rate to ensure some flow along the fibers or membranes. This flow along the fibers or membranes acts as a rinse or flush of the fibers or membranes to remove pyrogens.

A redundant pressure sensor 28e, conductivity sensor 30e and temperature sensor 32b are placed downstream from filters 122. A bypass valve 136 is located immediately after those sensors. If any of those sensors detect a reading out of range as indicated by step 214, e.g., if the saline solution is outside of a desired tolerance, valve 136 is opened at step 218 to force the fluid back to the input to the RO unit 52 along return line 66. That rejected saline is then used to check the performance of RO unit 52 using the percent rejection method described above.

### Solution Preparation—Lactated Ringers

System 10, besides saline, can also produce other types of injectable solutions, such as lactated ringers. Lactated ringers is a medication that is taken intravenously to supply water and electrolytes (e.g., calcium, potassium, sodium, chloride), either with or without calories (dextrose), to the body. Lactated ringers is also used as a mixing solution (diluent) for other intravenous medications.

The method to produce other types of solutions, such as lactated ringers, is basically the same as described above for producing saline. Here, product water exiting RO unit 52 flows through lubrication line 80 to pump 82, which to produce lactated ringer solution is referred to as an electrolyte pump 82. It should be appreciated that system 10 can produce NaCl solution and lactated ringers as desired.

Pump 82 is again in one embodiment a rotating, reciprocating ceramic piston pump. That type of pump requires lubrication, preferably with a clean or sterile lubricant such as product water exiting RO unit 52, which lubricates the shaft of ceramic pump 82. A peristaltic pump, diaphragm pump or gear pump is used alternatively for the electrolyte pump 82. In those cases, the flow path of system 10 would change accordingly, e.g., would not need a separate lubrication line 80. Water flowing through pump 82 via lubrication line 80 eventually reaches water receptacle 86.

Pressurized water from the RO flows via electrolyte pump 82, through line 80 to the water chamber 86. Water Chamber 86 provides a reservoir for incoming water. Water from chamber 86 reservoir flows to mix with a concentrated lactated ringers solution delivered via line 84. Water chamber 86 also provides a source of water for the NaCl cartridge 98, which produces NaCl solution via the switching of valves 94 and 96 as described above. To prevent stagnant areas from forming in the flow path, valves 94 and 96 and associated hydraulics are rinsed and disinfected after lactated ringers preparation. As an additional quality assurance procedure, system 10 senses whether or not an NaCl cartridge has been loaded into the system.

A lactated ringers concentrate connector 140 is provided downstream of cartridge fill valves 94 and 96. A female half of connector 140 is located downstream of the cartridge fill valves 96 and 94 and activates an automatic shutoff when the male portion of the connector is removed. A sensor is located on or adjacent to connector 140 to determine whether or not the male and female haves are connected together. That sensor may be an optical, reed, micro or any other switch that can sense the opening of the connection. System 10 uses such sensors in the holder for cartridge 98 and in the lactated ringers connector 140 to send a signal to the controller to determine whether system 10 is currently configured to produce saline or lactated ringers. If the sensors provide contradictory information (e.g. the cartridge sensor senses the NaCl cartridge 98 at the same time the lactated ringers fitting sensor senses that a container of lactated ringers is connected to system 10), system 10 will report an error to the operator.

An alternative method of setting system 10 to proportion NaCl solution or lactated ringers is to have the operator set the desired type of concentrate on a user interface for system 10, such as via a touch screen controller or via an electromechanical switch. The operator can manually engage or separate the lactated ringer connector 140, which in one preferred embodiment is accessible from outside of an enclosure 158 for system 10. Connector 140 fluidly connects system 10 to a container 138 of lactated ringer concentrate. While container 138 is illustrated as being connected fluidly to line 104 via connector 140, it is introduced alternatively into other suitable lines within system 10.

The lactated ringers concentrate within container 138 includes a concentrated solution of electrolytes, such as sodium (Na), chloride (Cl), calcium (Ca), potassium (K) and lactate. The concentrate is mixed proportionally so that it can be mixed at a desired ratio with purified water to produce a solution with the following approximate composition: Na at 130 mEq/l, K at 4 mEq/l, Ca at 2.7 mEq/l, Cl at 109 mEq/I and lactate at 28 mEq/l. Other concentrate solutions could also be employed to produce solutions such as a dextrose/NaCL.

A level control sensor 92a, such as an optical level sensor, reed switch or Hall effect switch with a float assembly, is provided at the top of the air trap 90. When sensor 92a senses that the water level in trap 90 is too low, the air trap vent valve 102 opens momentarily, while sprayer assembly 110 is under a vacuum. The vacuum pulls the concentrated lactated ringers solution from the lactated ringers concentrate container 138. If the liquid level in air trap 90 is too low, pump 82 is turned off temporarily. Air trap 90 is connected to the sprayer assembly 110 via the air vent valve 102 and flow restrictor 112. Sprayer 110 is thereby placed under a vacuum created by starved pump 106, which vents off any air bubbles from the liquid through air trap 100. When the level in the air trap 90 is restored to a proper or desired level, valve 102 closes and the pump 82 is restarted.

Pump 82 pumps a precise amount of lactated Ringers solution to mix with water in the tube just below water chamber 86. Inside this tube is a torturous path to thoroughly mix the water with the lactated solution and the solution flows to the sprayer assembly. Air removal pump 106 pulls the solution through an orifice in the sprayer assembly 110. Air removal pump 106 pumps at a rate higher then the flow through the sprayer orifice creating a nucleation site for bubbles that are bled off in air trap 100. The solution level in air trap 100 is controlled by a optical sensor at the top of air trap 100. This could also be a reed or hall effect switch with a float assembly.

Cartridge fill valves 94 and 96, which are used to produce the NaCl solution as discussed above are closed when system 10 is operated in lactated ringers mode. If a rinse is performed before or after the lactated ringer solution is produced, however, rinse water can be pumped through valve 96, through a bypass 142 around cartridge 98, and shunted through to valve 94. A cartridge holder for a dialysis machine having a similar bypass arrangement is described in commonly owned U.S. Pat. No. 6,036,858, the contents of which are hereby incorporated by reference.

The proportioning of the lactated ringers with water is accomplished by adjusting the electrolyte pump 82 with a feedback loop to achieve a particular conductivity. To that end, a conductivity sensor 30d is provided. Another approach is to volumetrically mix the solution, e.g., to mix a known volume or weight of cleansed water with a known volume or weight of electrolyte. Conductivity monitoring provides a confirmation of the solution mixture. The controller of system 10 monitors the conductivity output from sensor 30ed continuously. A thermistor, thermocouple or other type of temperature sensing device is provided (not illustrated) and used to compensate for any temperature changes in the electrolyte proportion calculation using conductivity measurements.

An electrolyte cartridge 198 could be used just as NaCl cartridge 98 is used via the manipulation of valves 94 and 96 as described above if only a single electrolyte is employed. A liquid concentrate provided within container 138 is used when lactated ringers or other type of solutions requiring multiple constituents or electrolytes is being produced. Concentrate would also be used to make dextrose NaCl. The concentrate within container 138 includes, for example, a solution that when mixed with water at the appropriate ratio produces a solution with the following approximate composition: Na at 130 mEq/l, K at 4 mEq/l, Ca at 2.7 mEq/l, Cl at 109 mEq/l and lactate at 28 mEq/l. The proportioning ratio of the above constituents can vary and can include more or less constituents than those described above, such as dextrose.

Those variations in concentrate formulation produce different lactated solutions. In addition, system 10 can use concentrates to form a dextrose NaCl solution or a pure dextrose solution, in which case a dextrose sensor (not illustrated) is provided. Concentrate 138 may also be sterile and/or pyrogen free to reduce the overall microbial burden in the system. When liquid concentrates are used, bypass 142 is used to bypass NaCl cartridge 98 or electrolyte cartridge 198.

In the lactated ringer or dextrose NaCl run state, concentrate pump 82 pulls concentrate 138 from its container through air trap 90. Concentrate pump 82 moves the solution to air trap 100 and sprayer air removal assembly 110, which mixes the solution in a torturous path. Sprayer assembly 110, as above, removes air by pumping the concentrate solution through a nozzle creating a nucleation site for bubbles that are bled off air trap 100. The liquid level in air trap 100 is controlled by an optical sensor 92b in combination with pump 106. As an alternative, micro switches, Hall effect and reed type switches may be used in combination with an air trap float.

The proportioning of the concentrate solution is achieved via the feedback loop that controls pump 82 to run until a desired conductivity is sensed by sensor 30d. If the conductivity rises too much, the pump is slowed. If the conductivity is lowered too much, the pump 82 speed is increased. Conductivity sensor 30d is therefore pivotal to the control of the proportioning of the lactated ringer/dextrose saline solutions in the illustrated embodiment. The conductivity monitoring confirms that the concentrate is mixed at a certain proportion with the purified water. If the temperature is constant, the concentrate is prepared properly, the conductivity should accurately reflect if the solution is mixed properly. Conductivity of the solution is monitored continuously and a thermistor is used to compensate for any temperature changes to ensure that electrolyte changes can be detected properly. As before, another method for proportioning the concentrate solution is by a volumetric mixing of the solution as described above for straight saline production.

The pump 114, pressure regulator 116 and bypass line 118 are provided to pump a pressure regulated concentrate solution from air trap 100 to a bubble test valve 120. Those components operate as described above in the NaCl description.

The integrity of ultrafilters 122 used with the concentrate solution can be tested using the bubble point test described above. Again, if one or more of the filters 122 fails the pressure decay test, the defective filter is changed prior to further use in saline production. As before, purge valve 134 and pump 132 enable a portion of the incoming concentrate solution to flow along the length of one of the two filters 122 to prevent the build-up of bacteria and/or endotoxin on the outside of the membranes located inside the filters.

A redundant pressure sensor 28e, conductivity sensor 30e and temperature sensor 32b are placed downstream from filters 122. A bypass valve 136 is located immediately after those sensors. If any of those sensors detect a reading out of range, e.g., if the concentrate solution is outside of a desired tolerance, valve 136 is opened to force the fluid back to the input to the RO unit 52 along return line 66. That rejected concentrate solution is then used to check the performance of RO unit 52 using the percent rejection method described above.

It should be appreciated that system 10 of FIG. 1 as well as the alternative systems shown below illustrate certain possible methods to accomplish the goal, namely, to provide an on-line portable source of injectable quality fluids. The specific flow path can be changed or varies by those of skill in the art without departing with the scope and spirit of the present invention. In addition, the valve function of every valve in the system in one embodiment is confirmed by conductivity measurements or other suitable method of confirming that the valves are functioning properly.

### Bag Filling

Referring again to FIG. 8, bag filling the saline or concentrate solution (hereafter collectively referred to as “injectable solution”) is accomplished using a pump 150 and one of a number of different possible volumetric control methods and types of flow metering devices, such as volumetric diaphragm type balancing chambers, mass flow meters, vortex shedding flow meters, turbine flow meters. One of the methods includes filling the bags via a flow balancing chamber 152, which is illustrated as an alternative component in system 10 of FIG. 1. Balancing chamber 152 operates with alternating valve pairs to deliver a precise volume of solution to one of the bags 154 by intaking simultaneously the same amount of fluid as indicated by step 216. The chamber includes a flexible membrane that moves back and forth to dispel and accept fluid on alternating sides of the membrane.

Another possibility is to use pump 150 and a volume flow sensor 156 to meter fluid into bags 154. A further alternative is to use pump 150 in combination with a scale or gravametric measurement to fill bags 154. In each of the alternatives, the bags 154 can be supported on all sides so that inlet pressure can be used to independently confirm that the bag is full. System 10 using any of the flow metering devices described herein meters into the sterile bags 154a to 154d (collectively bags 154 or generally bag 154) desired and precise amounts of an injectable quality solution.

While bags 154 reside on the outside of an enclosure 158 of system 10 for ease of access in one embodiment, it may be desirable to use a metering or weighing device that is placed on the inside of the enclosure 158, to protect same. In one preferred embodiment, a number of bags 154 are ganged or manifolded together, so that the number of connections is limited. It is important to note that the system is not limited to four bags, but can include any suitable numbers of bags, rows of bags, sizes of bags, etc.

Bags 154a to 154d can be filled individually, simultaneously or in any combination via pinch clamps 160a to 160d placed upstream of their respective bags 154a to 154d. Those clamps are connected fluidly to a manifold 162, which connects to an aseptic connector 164, such as a bulkhead connector, leading into the enclosure 158. Cartridge 98, 198 and the container for concentrate 138 in one preferred embodiment are located with respect to enclosure 158 so that they may be readily changed.

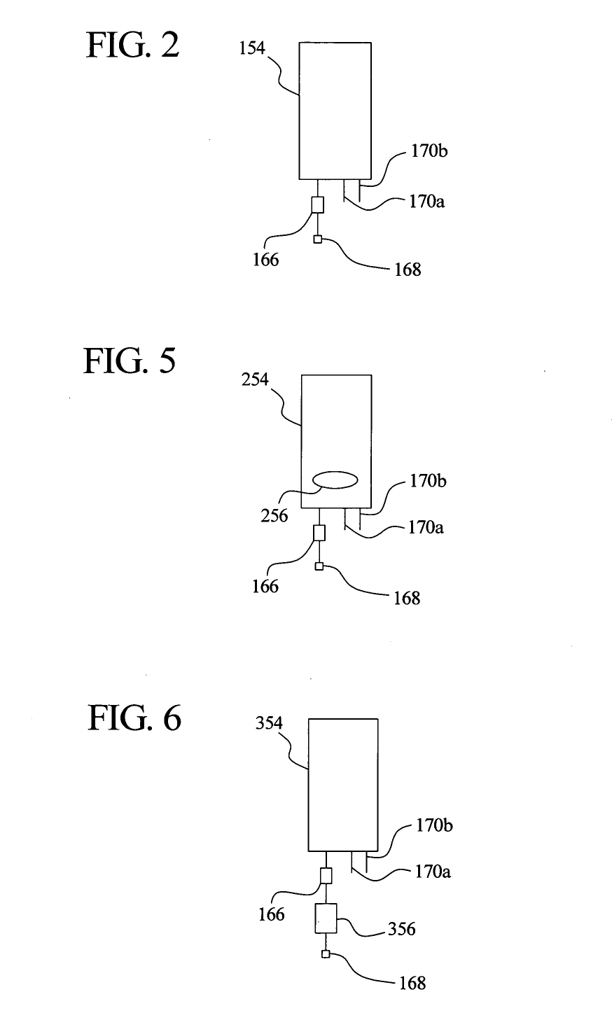

Referring now to FIG. 2, sterile bags 154 can be of any suitable size, such as one to two liters. Bags 154 in one embodiment include a 0.2 micron filter 166 located between the bag 154 and a connection 168 to the portable injectable solution system 10. When a series of ganged or manifolded bags 154 are provided as seen in FIG. 1, a single filter 166 can be provided on line 62 between the first and second bags 154a and 154b. In that way, the number of filters 166 is reduced but the additive solution going to any bag 154 necessarily flows through one of the filters 166. The first bag 154a is then discarded after it is filled as described below. Filter 166 protects against accidental contamination of the bag inlet when bag 154 is connected to system 10 or due to contaminants in line 162.

As seen at step 220, bags 154 also include standard intravenous bag connections 170a and 170b, which are used for delivery of the additive solution in a clinical environment. In one embodiment, the first bag of any set, e.g., bag 154a in FIG. 1 would not include connections for routine clinical use because that bag is provided to be discarded in case there is any contamination when bags 154 are first connected. Because the first bag is discarded and due to the configuration of filter 166 in the line 162, any bag contamination is flushed with clean solution from the instrument. Thereafter, all succeeding bags have one extra filtration step to ensure the microbial quality of the final solution.

As seen at step 222, bags 154 in one embodiment include a hard or extra edge with perforations between the two layers for use with an automatic feed mechanism. The feed mechanism delivers the correct amount of additive solution to one or more bags 154 simultaneously and one or more bags 154 sequentially. Bags 154 in one embodiment may be pre-labeled with a code for an optical scanner or a bar code to ensure that the bag is filled with the correct solution. That label could also be used with the normal product labeling for the bag. The feed mechanism may therefore include a printer or labeler, operable, as seen at step 224, with the controller of system 10, which is suitable for printing on plastic bags. The printer or labeler produces a print or a label (“collectively referred to as label”) having at least one of or any combination of: a lot code, the date of filling and expiration date.

Intravenous solution bags 154 are made of a thermoplastic, in one embodiment, such as vinyl. The opening through which bag 154 is filled is sealed by heating the bag at step 224 in the vicinity of the opening to a temperature sufficient to melt portions of the bag and then pressing the melted portions of the bag together as they cool to weld the opening shut. Several methods can be used to heat bags 154. One such method is radio frequency (“RF”) welding in which high frequency electromagnetic radiation is directed toward the bag to heat the plastic. Another method of sealing the bag is ultrasonic welding in which a portion of bag 154 is clamped between a sonic horn and an anvil. The horn vibrates against bag 154 at very high speeds (e.g., 20-40 kHz or more). As the horn vibrates, it moves toward and away from the bag and heats the bag, first at the outside surface and then further inward. Because the outside surface of the bag is heated first and the inside surface must be melted to weld the opening shut, bag 154 melts through its entire thickness during ultrasonic welding. Melting weakens the bag and prevents it from being suspended from above the weld. Therefore, bag 154 should be supported both below the weld to prevent it from rupturing and above the weld to prevent it from spilling.

### Disinfection

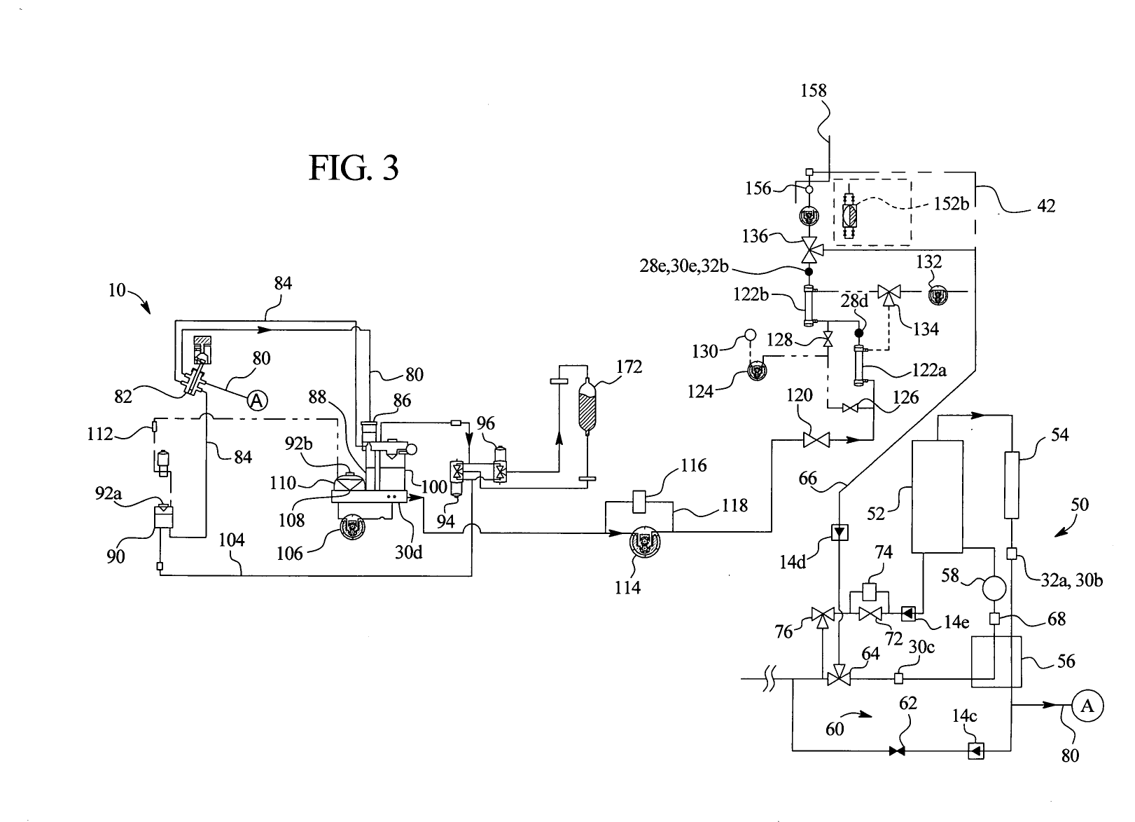

Referring now to FIG. 3, because system 10 is a point of use system, the system needs and includes periodic disinfection capabilities. FIG. 3 illustrates the portion of system 10 which forms the recirculation path. Notable missing is the pretreatment unit 20 and the bags 154. In an alternative embodiment, pretreatment unit 20 could also be flushed with a disinfectant.

In one embodiment, system 10 is rinsed free of injectable solution via a post-use flush cycle. With the flush cycle, the NaCl cartridge 98 or electrolyte cartridge 198 is replaced with a disinfect cartridge 172, which can be smaller than cartridges 98 or 198. In one embodiment, the cartridges are switched manually. In an alternative embodiment, cartridge 172 is used in combination with cartridge 98 or 198 and a suitable valve arrangement to enable one or the other to be selected at any given time for use. Cartridge 172 in one embodiment houses citric acid or actril, which is a hydrogen peroxide and peracetic acid-based disinfectant.

If citric acid is used, heater 54 heats the injectable solution to about 85° F. (29.5° C.) or greater for a specified period of time while mixed with citric acid. Such heated disinfectant provides a very high level of disinfection in removing bacteria, such as spore-forming bacteria, mold or other contaminants. Actril or other types of hydrogen peroxide/peracetic acid combinations are suitable for use as cold disinfectants.

### Controls/GUI

System 10 is controlled at the location of system 10 or controlled remotely. When at the location of system 10, the system is provided with a suitable control panel, monitor, touch screen interface, programmable logic controllers, control circuit boards and the like. System 10 is completely automated except possibly for the placement of the bags on the machines and the initiation of the bag-fill process. Alternatively, the bags can be moved, connected and disconnected automatically. If controlled remotely, system 10 is adapted to receive RF signals, RS-232 commands, RS-485 commands, internet commands or other type of suitable remote digital or analog signal remotely from an operator or central control station. A local operator is then used in one embodiment to load empty bags, connect the empty bags, disengage and unload full bags.

The monitor or touch screen displays outputs from various sensors, displays the number of units produced, etc. If a problem with system 10 occurs, the screen displays the source of the problem along with a suggested solution in one embodiment. The control scheme is operable with minimal training by a user, who may or may not be familiar with aseptic technique. The components of system 10 described herein each include built-in redundancy in one embodiment. System monitoring and controlling will be independent in an embodiment so that a failure of a certain component or function does not effect the operation and integrity of the other components and functions of system 10.

### Alternatives

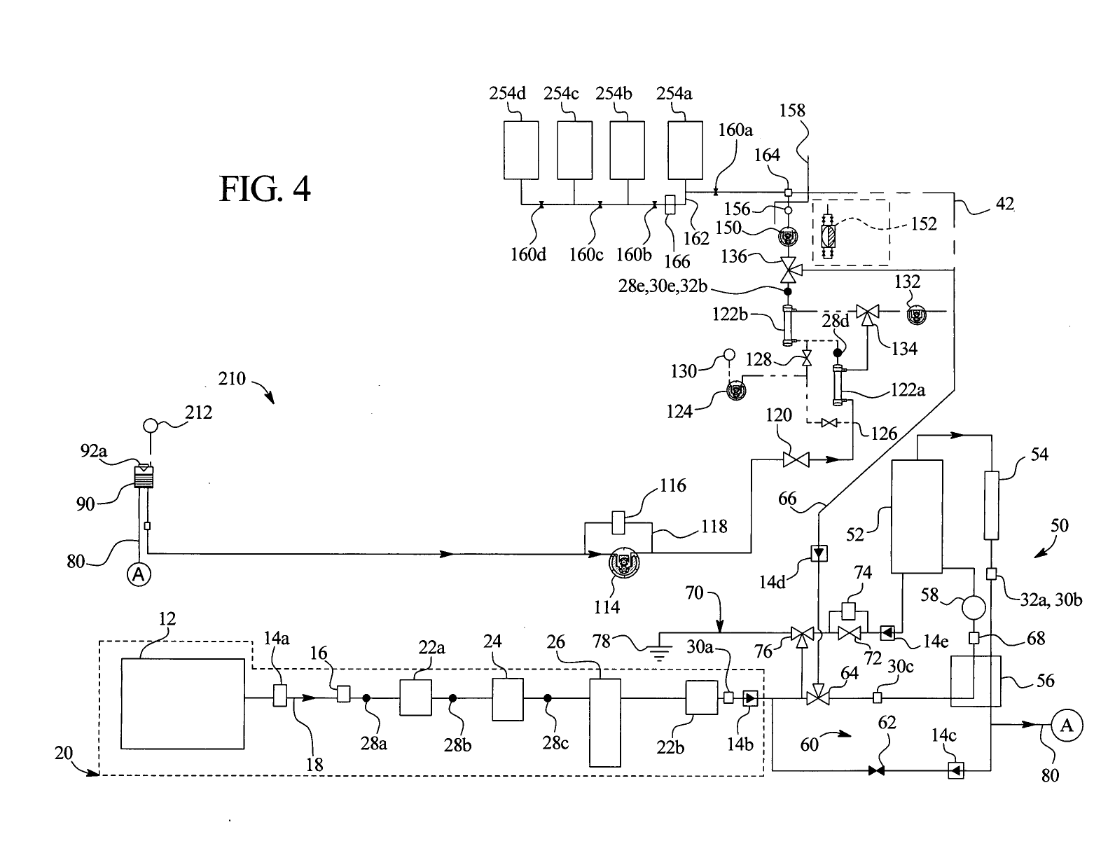

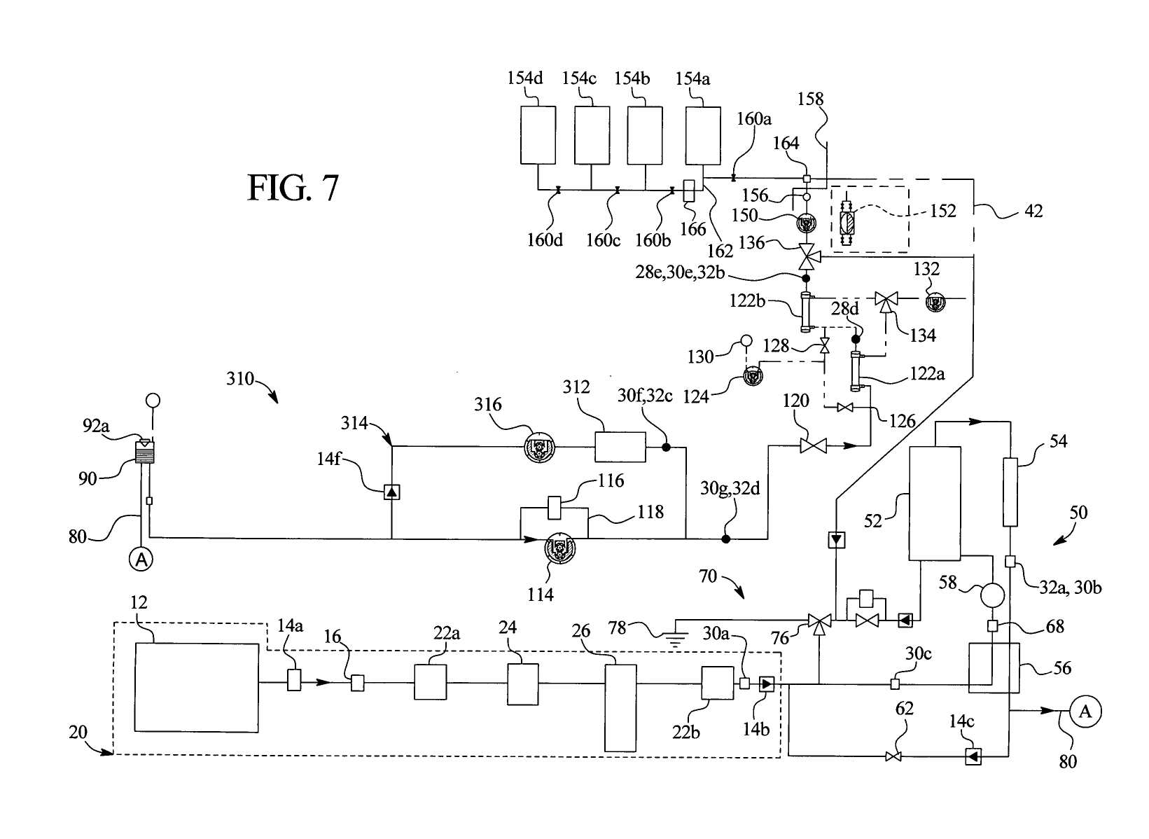

Referring now to FIGS. 4 and 5 an alternative system 210 is illustrated. System 210 includes many of the same components as system 10 of FIGS. 1 and 2. Each of those same components is numbered the same in FIGS. 4 and 5 as in FIGS. 1 and 2 and the description and alternatives for those element numbers described above is applicable to the like numbers FIGS. 4 and 5.

System 210 however does not employ the cartridge proportioning components for NaCl or electrolyte injection described in connection with system 10. In particular, pump 82, 182, air vales 102, pump 106, sprayer air removal assembly 110, optical sensor 92b, receptacle 86, air trap 100, conductivity sensor 30d, cartridge fill valves 94 and 96, cartridge 98, 198 and bypass 142 are removed. Air trap 90, optical sensor 92a and a vent filter 212, such as a 0.2 micron vent filter, are retained.

In system 210, a predefined amount of NaCl, electrolyte or pre-sterilized concentrate (collectively “additive 256”) is provided inside of alternative bags 254a to 254d (collectively “bags 254” or generally “bag 254”). In one embodiment, additive 256 is sterile or injectable NaCl, hypertonic NaCl or any of the other additives described herein. Bags 254 each include a filter 166, such as a 0.2 micron filter, a connector 168 and standard IV bag connectors 170a and 170b described above.

System 210 produces injectable water, rather than solution, for injection into alternative bags 254, where the water is mixed with one or more additive 256. Additive 256 is provided in an amount proportioned to the amount of injectable water pumped into bag 254 to produce a desired solution. Bag 254 can be of any suitable size, such as one to two liters. System 210 thereby produces bags 254 of injectable quality solution utilizing sterile or injectable additives and the injectable quality water.

In one embodiment, an agitator is used to ensure that the additive 256 is mixed properly inside bag 254 with the injectable quality water. The agitator can be sized, configured and situated to agitate only a single bag 254 or multiple bags 254 at once and can be integral with or separate from the enclosure 158 or remainder of system 210.

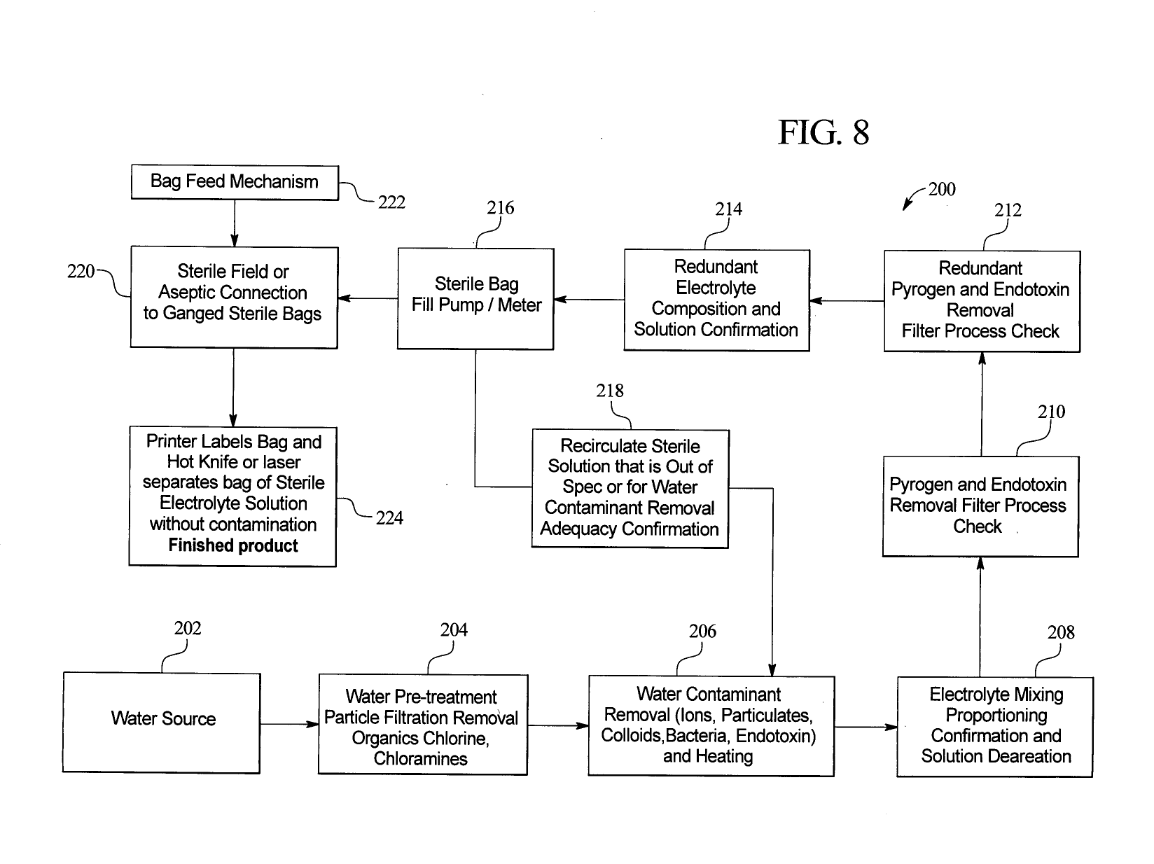

Referring now to FIG. 6, system 210 is additionally operable with alternative bag 354. Bag 354, instead of holding an amount of additive 256 internally, employs an additive pack 356, such as a PrisMedical™ NaCl Delivery Pack, in the line extending from the bag receptacle that also includes filter 166 and connector 168. Bag 354 can also include connectors 170a and 170b as illustrated.

With bag 354, system 210 operates as described above, producing injectable quality water, not solution, for delivery to bag 354. The solution, including NaCl, dextrose, any of the concentrate materials discussed herein and any combination thereof, is produced as the purified water flows past connector 168, through additive pack 356, through filter 166 and into bag 354. System 210 operates differently with bag 254 of FIG. 5, which mixes the solution initially inside bag 254. The agitation described above can also accompany the use of bag 354 to help ensure proper mixing.

System 210 operating with either bags 254 or 354 can produce any of the injectable additive solutions described above such as saline, dextrose NaCl, lactated ringers and the like.

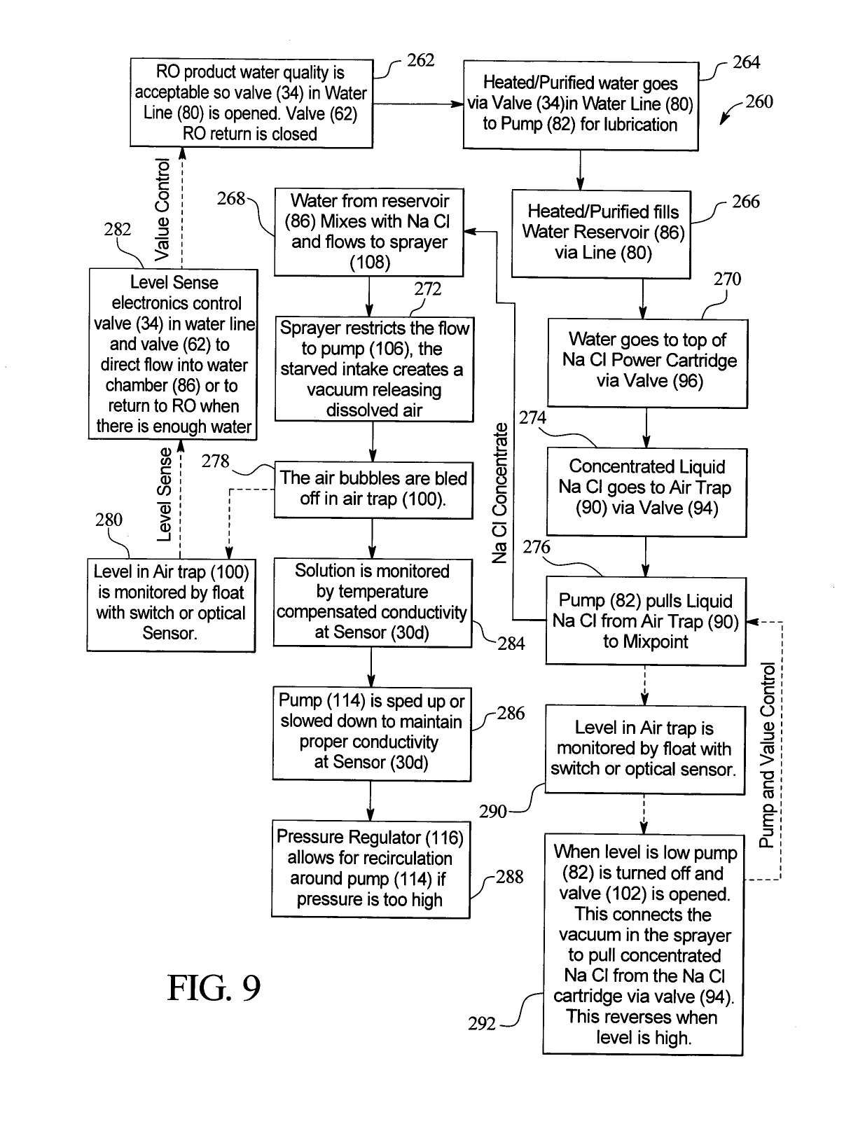

Referring now to FIG. 7, another alternative system of the present invention is illustrated by system 310. System 310 employs an additive pack 312, such as a PrisMedical™ drug delivery pack, as does system 210 in operation with bag 354 in FIG. 6. Pack 312, however, is located in the water purification flow path. In the illustrated embodiment, pack 312 is located downstream from airtrap 90 and upstream from bubble test sensor 120. It is possible that pack 312 is located in alternative positions along the water purification flow path.

In system 310 of FIG. 7, pack 312 is a relatively large drug delivery pack (e.g., containing two to three kilos of electrolyte) with respect to pack 356 of FIG. 6. It is advantageous from a cost and feasibility standpoint to place a single pack 312 in the purification line rather than to provide smaller packs 356 with each bag 354 as is done in FIG. 6. It is also contemplated to run multiple redundant additive packs 312 in series in case one pack fails or is depleted during operation. Conductivity sensors 30f and 30g located downstream from pack 312 can detect if pack 312 is not operating properly and signal a suitable alarm to the control panel or monitor, after which an audio or visual message can be displayed to change the pack 312.

Pack 312 is provided in a recirculation loop 314 along with a pump 316, check valve 14f, conductivity sensor 30f and temperature sensor 32c in the illustrated embodiment. Water that is substantially purified by pretreatment unit 20 and RO unit 52 is pumped via pump 316 through additive loop 314 and one or more additive pack 312 until conductivity sensor 30f and/or 30g, operating in conjunction with temperature signals from sensors 32c and 32d, respectively, indicate that the solution is at a desired concentration.

Until the solution is ready, bubble test valve 120 remains closed. During additive circulation, regulator 116 and pump 114 operating through loop 118, ensure that the pressure within loop 314 does not exceed a pressure established by regulator 116. When the solution reaches the desired additive concentration, bubble test valve 120 is opened, after which the properly concentrated injectable fluid is allowed to flow through ultrafilters 122 and ultimately to the bags 154 described above in connection with system 10 of FIGS. 1 and 2. It is advantageous that the mixed fluid runs through ultrafilters 122, unlike the system 210 operating with bags 254 or 354, which provide a extra level of redundancy. The solution is also mixed properly before reaching bags 154, eliminating the need for the above-described agitation. When conductivity sensors 30f and/or 30g sense that the concentration is out of range, bubble test valve 120 is closed and the above-described cycle is repeated.

Pack 312 can be used to produce any of the injectable additive solutions described above such as saline, dextrose NaCl, lactated ringers and the like. Pack 312 in one preferred embodiment is located with respect to enclosure 158 so that it may be readily changed.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. An injectable fluid preparation system comprising:
an inlet configured to be coupled to a source of water;
a filtering device operable to at least partially purify water flowing from the source;
an additive supply in fluid communication with the filtering device, the supply supplying at least one additive to the at least partially purified water to form an additive solution;
at least one sensor having an output indicative of a proportion of the additive in water;
a housing supporting at least the filtering device and the sensor, the housing including a connector in fluid communication with the additive solution; and
a container connected fluidly to the connector, the container receiving an amount of the additive solution and configured to store the additive solution for later use.

2. The injectable fluid preparation system of claim 1, which is operable to accept water from a source selected from the group consisting of: a water tap, a container of water and a natural water source.

3. The injectable fluid preparation system of claim 1, wherein the filtering device is selected from the group consisting of: a particulate filter, a carbon filter, a deionization unit, a reverse osmosis unit and an ultrafilter.

4. The injectable fluid preparation system of claim 1, wherein the filtering device is a first filtering device and is located upstream from the additive supply, and which includes a second filtering device, the second filtering device having at least one characteristic selected from the group consisting of: (i) being located downstream from the additive supply; (ii) being located between the connector and the container; (iii) being coupled operably to a pressure testing apparatus; (iv) being coupled operably to a control unit that monitors a rate of decay of pressure in the second filtering device; (v) being an ultrafilter; and (vi) including a membrane that has a pore size from about 10 to about 1000 Angstroms.

5. The injectable fluid preparation system of claim 1, wherein the filtering device includes a reverse osmosis unit and which includes at least one of: (i) a water pretreatment unit located upstream of the reverse osmosis unit; (ii) at least one reject fluid line in communication with the reverse osmosis unit; and (iii) a fluid line enabling a portion of the additive solution to be recirculated back to the reverse osmosis unit.

6. The injectable fluid preparation system of claim 5, wherein the pretreatment unit includes at least one filter of a type selected from the group consisting of: a particulate filter, a carbon filter and a deionization unit.

7. The injectable fluid preparation system of claim 5, which includes at least one additional filter located downstream from the reverse osmosis unit.

8. The injectable fluid preparation system of claim 5, wherein the reject fluid is at least one of: (i) purified with respect to water from the source; and (ii) rejected if an output of filtered water from the reverse osmosis unit exceeds a downstream demand for the filtered water.

9. The injectable fluid preparation system of claim 5, wherein a conductivity measurement of the recirculated portion is measured against a conductivity measurement of the RO unit's output to determine a level of performance of the RO unit.

10. The injectable fluid preparation system of claim 5, wherein a percent rejection method is employed to determine a level of performance of the RO unit, and wherein an at least one of an alarm and a system shut down is triggered if the percent rejection is outside of an acceptable level.

11. The injectable fluid preparation system of claim 1, wherein the additive supply is of a type selected from the group consisting of: an additive cartridge, a liquid concentrate container and an in-line additive delivery pack.

12. The injectable fluid preparation system of claim 1, wherein the additive is selected from the group consisting of: NaCl, dextrose, sodium, potassium, calcium, chlorine, lactate, a non-sterile additive and any combination thereof.

13. The injectable fluid preparation system of claim 1, wherein the additive supply includes at least one of: (i) an air separation device that removes air trapped in the at least partially purified water; (ii) a pump requiring a priming fluid, wherein the at least partially purified water is used as the priming fluid; and (iii) a recirculation loop that enables the at least partially purified water to be recirculated until being proportioned with the additive properly according to the sensor.

14. The injectable fluid preparation system of claim 1, wherein the sensor includes a conductivity sensing device.

15. The injectable fluid preparation system of claim 1, wherein the sensor is a first sensor and which includes a pressure sensor that is operable to provide a pressure decay signal used to evaluate the integrity of the filtering device.

16. The injectable fluid preparation system of claim 1, wherein the amount of the additive solution is a predefined amount, and which includes a flow metering device that meters the predefined amount of additive solution to the container.

17. The injectable fluid preparation system of claim 1, which includes multiple containers manifolded in fluid communication with the additive solution and a valve arrangement operable to selectively allow at least one of the containers at a given time to be filled with additive solution.

18. The injectable fluid preparation system of claim 17, wherein one of the containers is discarded after initial filling and which includes a filter between the discarded container and the remaining containers.

19. The injectable fluid preparation system of claim 1, which includes a disinfectant injector operable to disinfect at least the additive supply of the system, the disinfectant including a substance selected from the group consisting of: high temperature water, diluted acid, citric acid, hydrogen peroxide, peracetic acid and any combination thereof.

20. The injectable fluid preparation system of claim 19, which includes a controller operable to perform at least one of the tasks selected from the group consisting of: (i) automatically add the disinfectant in a desired proportion; and (ii) rinse the disinfectant from the additive supply automatically.

21. The injectable fluid preparation system of claim 1, wherein the container has at least one property selected from the group consisting of: (i) being at least substantially sterile; (ii) being made of a biocompatible thermoplastic; (iii) being made of a flexible material; (iv) having a code to identify the additive solution; and (iv) being operable with a labeling device that labels the container.

22. An injectable fluid sterilization system comprising:
a pretreatment unit that filters water from a source;
a filtering device that further filters the water from the pretreatment unit;
an additive supply in fluid communication with the filtering device, the supply supplying at least one additive to water exiting the filtering device; and
a delivery portion operable to meter additive solution exiting the additive portion to a container, the container configured to store the additive solution for later use.

23. The injectable fluid sterilization system of claim 22, which is operable to accept water from a source selected from the group consisting of: a water tap, a container of water and a natural water source.

24. The injectable fluid sterilization system of claim 22, wherein the pretreatment unit includes at least one filter of a type selected from the group consisting of: a particulate filter, a carbon filter and a deionization unit.

25. The injectable fluid sterilization system of claim 22, wherein the filtering device is selected from the group consisting of: a particulate filter, a carbon filter, a deionization unit, a reverse osmosis unit and an ultrafilter.

26. The injectable fluid sterilization system of claim 22, wherein the filtering device is a reverse osmosis (“RO”) unit, and which at least one reject fluid line in communication with an inlet of the RO unit, the reject fluid line having at least one characteristic selected from the group consisting of: (i) feeding the RO unit with a supply that has already passed through the RO unit to thereby lessen an overall load placed on the RO unit; (ii) being configured to extend from the RO unit; and (iii) being configured to extend from a location positioned downstream from the additive flow portion.

27. The injectable fluid sterilization system of claim 22, wherein the additive supply includes at least one of: (i) an additive cartridge, (ii) a liquid concentrate container, (iii) an in-line additive supply pack, (iv) a recirculation loop operable to enable the at least partially purified water to be recirculated until being proportioned properly with the additive, and (v) a volumetric metering device.

28. The injectable fluid sterilization system of claim 22, wherein the additive is selected from the group consisting of: NaCl, dextrose, sodium, potassium, calcium, chlorine, lactate and a combination thereof.

29. The injectable fluid sterilization system of claim 22, wherein a proportion of additive to water is controlled via conductivity measuring of the additive solution.

30. The injectable fluid sterilization system of claim 22, which includes multiple containers manfolded in fluid communication with the additive solution and a valve arrangement operable to selectively allow at least one of the containers at a given time to be filled with additive solution.

31. The injectable fluid sterilization system of claim 22, wherein the container has a property selected from the group consisting of: (i) being at least substantially sterile; (ii) being made of a biocompatible thermoplastic; (iii) being made of a flexible material; (iv) having a code to identify the additive solution; and (iv) being labeled with at least one of a lot code, a date of filling and an expiration date.

32. The injectable fluid sterilization system of claim 22, further comprising a pump, in fluid communication with the filtering device, to pump the additive solution into the filtering device to aid in cleansing the filtering device.

33. An injectable fluid preparation system comprising:
an inlet configured to be coupled to a source of water;
a filtering device operable to at least partially purify water flowing from the source;
a metering device operable to meter an amount of injectable quality water to a container; and
an additive provided downstream of the metering device, the additive mixing with the injectable quality water to produce an injectable quality solution that is stored in the container, the container configured to store the additive solution for later use.

34. The injectable fluid preparation system of claim 33, wherein the additive has at least one characteristic selected from the group consisting of: (i) being supplied inside the container; (ii) being supplied outside the container; (iii) including NaCl; (iv) including dextrose; (v) including sodium; (vi) including potassium; (vii) including calcium; (viii) including chlorine; and (ix) including lactate.

35. The injectable fluid preparation system of claim 33, which is operable to accept water from a source selected from the group consisting of: a water tap, a container of water and a natural water source.

36. The injectable fluid preparation system of claim 33, wherein the filtering device is selected from the group consisting of: a particulate filter, a carbon filter, a deionization unit, a reverse osmosis unit and an ultrafilter.

37. The injectable fluid preparation system of claim 33, wherein the filtering device includes a reverse osmosis (“RO”), unit and which includes at least one of: (i) a water pretreatment unit located upstream of the RO unit; (ii) at least one additional filter located downstream from the RO unit; and (iii) at least one reject fluid line in communication with the reverse osmosis unit.

38. The injectable fluid preparation system of claim 33, wherein the pretreatment unit includes at least one filter of a type selected from the group consisting of: a particulate filter, a carbon filter and a deionization unit.

39. The injectable fluid preparation system of claim 38, wherein the reject fluid line includes at least one characteristic selected from the group consisting of: (i) extending from the reverse osmosis unit and (ii) producing reject fluid that is purified with respect to water from the source.

40. The injectable fluid preparation system of claim 33, wherein the metering device is a balanced flow chamber that outputs the amount of fluid to the container by intaking the same amount of fluid.

41. The injectable fluid preparation system of claim 33, which includes multiple ones of the containers manifolded in fluid communication with the solution, and a valve arrangement operable to selectively allow at least one of the containers at a given time to be filled with the solution.

42. The injectable fluid preparation system of claim 33, which includes multiple containers manifolded in fluid communication with the injectable solution and at least one of: (i) a feeder mechanism to fill each container and (ii) an apparatus operable to seal each container after it is filled.

43. The injectable fluid preparation system of claim 33, wherein the sealing apparatus includes a laser apparatus or a hot knife.

44. An injectable fluid preparation method performed by an apparatus, the method comprising:
accepting water from a source;
purifying the water to an injectable quality;
adding at least one additive to the injectable quality water to create an injectable quality solution; and
metering portions of the injectable quality solution to individual containers.

45. The injectable fluid preparation method of claim 44, wherein purifying the water to an injectable quality includes at least one of: (i) flowing the water through at least one filter provided by the apparatus and (ii) recirculation fluid downstream of the filter back to an input of the filter.

46. The injectable fluid preparation method of claim 44, wherein adding at least one additive to the injectable quality water includes at least one of: (i) flowing the water through an additive source used in connection with the apparatus; (ii) flowing the water through an additive recirculation loop provided by the apparatus until a desired amount of the additive is added to the water; and (iii) metering the portions though an additive source.

47. The injectable fluid preparation method of claim 44, wherein creating the injectable quality solution includes controlling the conductivity of the solution.

48. The injectable fluid preparation method of claim 44, which includes configuring the container to store the injectable quality solution for later use.

49. The injectable fluid preparation method of claim 44, which includes at least one additional step selected from the group consisting of: (i) disinfecting at least a portion of the apparatus; (ii) labeling at least one of the individual containers; and activating a flow path to shunt to rinse residual powder from the apparatus.

50. An injectable fluid preparation method performed by an apparatus, the method comprising:
accepting water from a source;
purifying the water to an injectable quality;
metering portions of the injectable quality water to individual containers; and
supplying the containers with an additive that mixes with the injectable quality water to produce an injectable quality solution.

51. The injectable fluid preparation method of claim 50, wherein purifying the water to an injectable quality includes flowing the water through at least one filter provided by the apparatus.

52. The injectable fluid preparation method of claim 51, which includes at least one additional step selected from the group consisting of: (i) recirculation fluid downstream of the filter back to an input of the filter; (ii) configuring the container to store the injectable quality solution for later use; (iii) disinfecting at least a portion of the apparatus; and (iv) labeling at least one of the individual containers.

53. A filter performance level determination method for an injectable fluid preparation system, the method comprising:
measuring the conductivity of an output flow of a filter;
reticulating a portion of the output through the filter;
measuring the conductivity of the portion after being flowed through the filter; and
using a percent rejection method according to the equation, % rejection=1−(output flow conductivity/recirculated output flow conductivity)×100, to determine the performance level of the filter.

54. The method of claim 53, wherein the filter is a reverse osmosis unit.
